(19) United States
(12) Patent Application Publication
(10) Pub. No.: US 2024/0156565 A1
(43) Pub. Date: May 16, 2024

(54) SURGICAL STERILE FIELD

(71) Applicant: SURGICAL DESIGN CORPORATION, Armonk, NY (US)

(72) Inventor: William Banko, Armonk, NY (US)

(73) Assignee: SURGICAL DESIGN CORPORATION, Armonk, NY (US)

(21) Appl. No.: 17/984,780

(22) Filed: Nov. 10, 2022

(51) Int. Cl. A61B 90/40; A61B 46/10; A61G 10/02

(52) U.S. Cl. CPC A61B 90/40; A61B 46/10; A61G 10/02; A61B 2046/205

(57) ABSTRACT

A surgical sterile field system includes an inflatable flexible enclosure that is transparent in at least part of its expanse and a pair of gloves that are hermetically formed and sealed on a wall of the enclosure to allow access to an inside of the enclosure near a surgical site. Further, the system includes an incision site of a size corresponding to the required surgical site. The site has an adhesive at least surrounding its exterior periphery that is attachable to the skin of the patient adjacent the surgical sight and a cover over the incision site adhesive which can be removed prior to use. The enclosure at the incision site can be cut by a surgical instrument to expose the surgical site.

### FIELD OF THE INVENTION

The present invention relates to a surgical sterile field. More specifically, the present invention relates to a microsurgical sterile field that is compact, portable, disposable and cost-effective.

### BACKGROUND OF THE INVENTION

In efforts to reduce cost associated with surgeries as well as to provide patients with more comfort, an increasing number of physicians have been practicing office-based surgeries, or OBS. OBS are surgical or invasive procedures performed in a location other than a hospital or ambulatory surgery center. In an OBS the procedures, which may include cataract surgery, retina procedures, refractive lens exchange and oculoplastic surgery, are performed in a suite located within the physician's office and utilize minimal to moderate anesthetics. However one major issue in practicing in an OBS is the risk of contamination.

In order to prevent contamination, the operating room needs to be continuously cleaned and disinfected to create a sterile environment. The surgeon and assistants must scrub their hands meticulously with disinfectant, and wear a sterile gown, gloves, a disposable mask, cap and disposable shoe coverings. Further, the patient must be extensively covered with sterile drapes. In case of an eye surgery, the eye must be draped with a special sterile cover for intraocular surgery (cataract or vitrectomy). Further, all surgical instruments must be sterile, e.g., by autoclaving them with high pressure steam. These are challenging, especially for those physicians who do not enjoy the same resources as large medical facilities.

Various efforts have been made to help surgeons practice OBS more easily. For example, US Patent Application Publication No. 2015/0238264 discloses a sterile ophthalmic surgical drape system that can provide a sterile operating field for ophthalmic medical procedures. The sterile ophthalmic surgical drape system comprises a drape, a collapsible frame apparatus, a hole of sufficient size for a microscope, a fan, a filter, air directors and slits. The drape system may be configured to be used in conjunction with a surgical tray with presterilized instruments. However, in this drape system, there is no disclosure of how the surgeon can get his hands into the operating site without breaching the sterile environment, except by reverse air flow. Also, the drape does not form a complete sterile enclosure.

Another example is US Patent Application Publication No. 2021/0220070 that discloses a portable surgical system including a sterile enclosure and a plurality of sterile sleeves made of a composite-material. However, while it is called “portable,” the surgical system still requires “rigid frame” structure. Further, in this system, the medical instruments are transferred into the enclosure via material ports that “may be opened and closed by various means, such as zippers, magnetic strips, hook-and loop fasteners.” These ports may cause contamination by introducing nonsterile air full of many particles, bacteria, and viruses, into the container.

The present invention has been made in view of the above-described circumstances in order to provide a surgical sterile field that is compact and provides an effective sterilized surgical site in a cost-effective manner without relying on a rigid frame structure or requiring heavy equipment such as an electric pump, while providing a sterile way for the surgeon's hands to reach the surgical site.

### SUMMARY OF THE INVENTION

The present invention is directed to a sterile surgical field system. When used for eye surgery, e.g., cataract removal, it includes an inflatable flexible enclosure that is transparent in at least part of its expanse and a pair of gloves that are hermetically formed on a wall of the enclosure to allow access for a surgeon's hands inside of the enclosure at the surgical site. A transparent portion of the enclosure is attached, e.g., by adhesive, to the objective lens of a microscope used by the surgeon during the operation. The system also includes an attachment portion in the form of an eye opening in the enclosure adjacent the patient's eyes. This eye opening has an adhesive along its outer peripheral edge and an exterior removable cover. A further entrance opening is provided in the enclosure through which cables for powering a surgical instrument such as an ultrasonic phacoemulsification surgical instrument, as well as irrigation and aspiration tubes.

The system is provided at a physician's office with the enclosure collapsed and already sterilized and in a sealed package. If the surgical instrument is of the disposable type, it may be pre-sterilized and provided within the enclosure with its power and fluid tubes sealed in the wall of the enclosure. The transparent portion of the enclosure is fastened to the objective lens of the microscope by the adhesive provided and the enclosure is inflated. During or after inflation, the eye opening is sealed to the skin of the patient around the eye or eyes. In such a state, the interior of the enclosure is sterile and the rest of the operating room need not be. The surgeon can put his or her hands in the gloves, pick up the surgical instruments and perform the surgery without contaminating the surgical field. Materials for bandaging the surgical wound that have been presterilized and placed in the enclosure before the procedure can then be used to bandage the patient. At that point, the enclosure can be detached from the microscope and from the skin of the patient. It and its contents, where disposable instruments are used, can then be thrown away.

This procedure for eye surgery can be expanded for use in surgical procedures on other parts of the body. In that setting or in eye surgery, additional instruments may be introduced in to the sterile enclosure. An instrument tray with the other instruments pre-sterilized has an adhesive layer over the top of the tray and a cover release layer over the adhesive. The cover layer is removed ant the tray stuck to the enclosure by the adhesive. In order to reach the instruments, the surgeon cuts through the enclosure and the adhesive layer.

Not only is the sterile surgical field of the present invention useful for surgery in doctor's offices, as opposed to hospitals and OBS offices, it can also be used for eye surgery or general surgery in disaster relief operations, or a battle field, or traffic accident cases. In these situations, every minute used in transporting a trauma patient to a hospital can be a matter of life and death. By using the surgical sterile field system of the present invention, procedures like suturing major arteries or veins to stop hemorrhaging can be performed at the disaster location before transporting the patient to the hospital for complete medical care. In addition, since the surgical sterile field system does not require any rigid frame structure, it is easy to carry and is by no means cumbersome.

The microsurgical sterile field embodiment of the present invention provides ophthalmologists with significant benefits because of its reduction in cost and infection rates. In particular, for those who practice OBS, the microsurgical sterile field of the present invention offers an ideal clinical setting since it makes surgical procedures more efficient, while eliminating all of the time, materials and cumbersome process needed to disinfect the operating room and sterilize the surgical gowns and drapes. The ecological (energy) footprints are also expected to be significantly reduced as a result.

### BRIEF DESCRIPTION OF THE DRAWINGS

These and other features and advantages of the present invention will become more readily appreciated when considered in connection with the following detailed description and appended drawings, wherein like designations denote like elements in the various views, and wherein:

FIG. 1 is a side view of a first embodiment of the present invention, in the form of a microsurgical sterile field system for ophthalmological procedure;

FIG. 2 is a plan view of the elements of the first embodiment of the present invention;

FIG. 3A shows an enlarged view of the entry port and FIG. 3B shows the detailed structure of the entry port;

FIG. 4A is a plan view of the enclosure showing the addition of two types of attachment portions for the first embodiment of the present invention and FIG. 4B is an enlarged side view of the attachment portion formed on the enclosure.

FIG. 5A is a perspective view of an instrument tray used in the surgical sterile field system of the present invention and FIG. 5B is a perspective view of the instrument tray in which the covered layer is peeled to expose the adhesive layer;

FIGS. 6A-6D illustrate the process of making the instruments in the instrument tray accessible to the surgeon in the enclosure of the surgical sterile field system, wherein FIG. 6A shows the cover layers of the attachment portion and the instrument tray removed to expose the adhesive layers, FIG. 6B shows the instrument tray attached to the enclosure; FIG. 6C shows a surgeon cutting the enclosure wall and the tray cover simultaneously, and FIG. 6D shows that the medical instruments are accessible;

FIG. 7 shows a sterile bag that is configured to enclose the instrument tray;

FIG. 8 shows a sterile bag that is configured to enclose the surgical sterile field system of the present invention;

FIG. 9 is a perspective view of a modified version of the first embodiment for use in a general surgical procedure; and

FIG. 10 illustrates the process of attaching the attachment portion of the balloon to the skin of the patient at incision site in general surgery;

FIG. 11 is a third embodiment of the present invention; and

FIG. 12 illustrates the work tip of a phacoemulsification handpiece with a laser energy source.

### DETAILED DESCRIPTION OF THE INVENTION

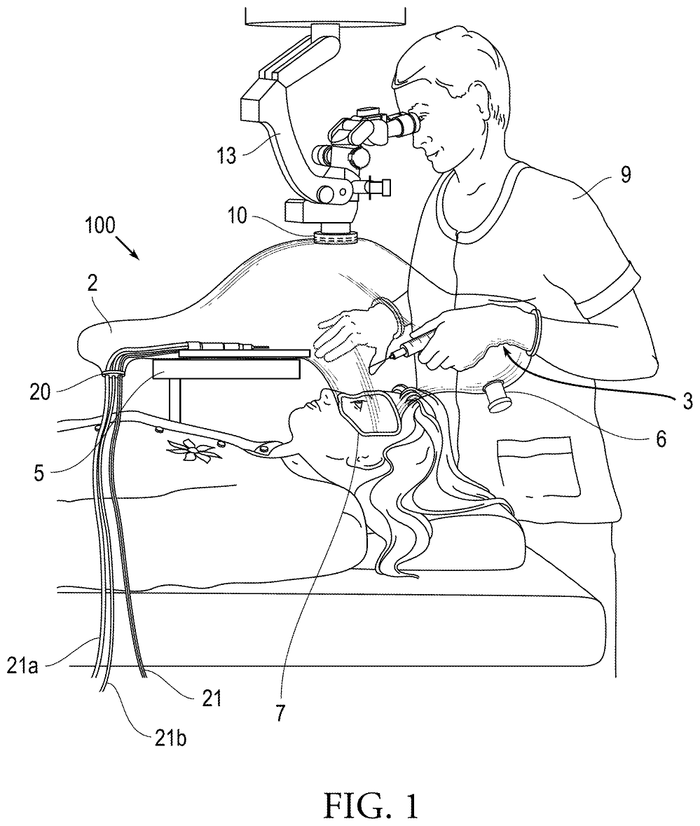

FIG. 1 shows a microsurgical sterile field system 100 of a first embodiment of the present invention for performing an ophthalmological operation. The surgical sterile field system 100 comprises an inflatable enclosure 2 that is made of flexible and inflatable materials, e.g., plastic. It has at least one pair of integral sterile gloves 3 used by a surgeon. The sterile gloves 3 are hermetically sealed to the wall of the enclosure 2 with their rims sealed against the outside environment, but so as to allow for access to the inside of the enclosure 2 by the hands of the surgeon.

FIG. 1 further shows a surgeon 9 performing an ophthalmological operation while viewing the incision site 7 through a microscope 13 attached to the microscope connection 10. The enclosure 2 is flexible enough to allow the objective lens of the microscope 10 to be aligned with the incision site 7 at the patient's eye. In a particular version, the enclosure may include a clear window 11 at connection 10 to improve the surgeon's view.

The microsurgical sterile field system 100 is simple and requires that only a small area be sterile compared to a traditional ophthalmological procedure. For instance, no disposal drapes or gowns are required. Accordingly, the microsurgical sterile field system 100 of the present invention makes it possible to efficiently perform an ophthalmological operation in a cost-effective and environmentally friendly manner, while minimizing the risk of contamination. This includes performing the operation in a doctor's office as opposed to a hospital surgical room or even an OBS suite.

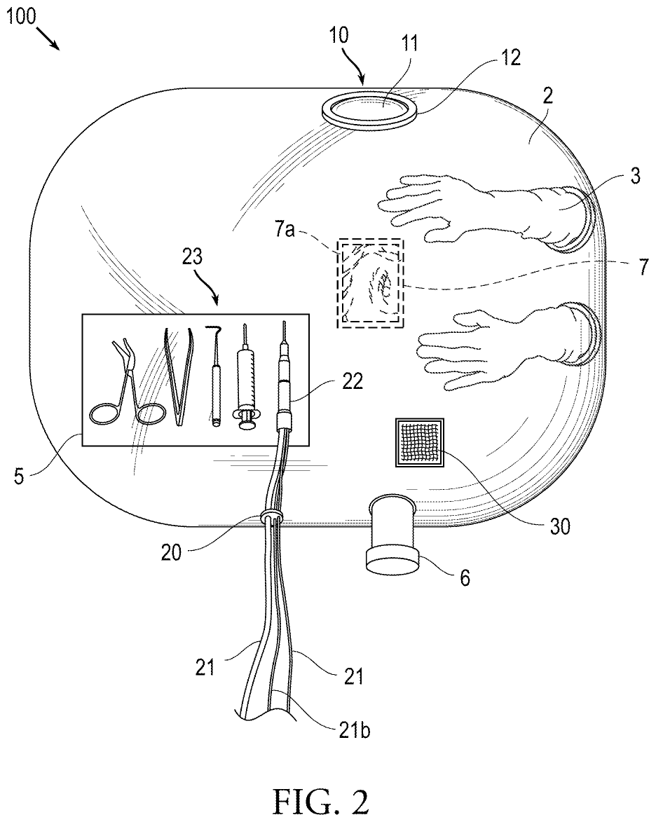

FIG. 2 is a plan view of the enclosure of FIG. 1. It shows the microscope connection 10 and a cable port 20. The microscope connection 10 comprises the view window 11 that is made of clear glass or hard plastic and a rim 12 surrounding the view window 11. The rim 12 is configured to attach to the microscope objective, e.g., by an adhesive. The cable port 20 is formed on the wall of the enclosure 2 so that the power cord 21, irrigation 21a and/or evacuation tubes 21b of a surgical instrument 22 can be can extend outside of the enclosure 2 through a seal. The surgical instrument and other instruments 23 needed for the procedure and located in a surgical tray 5 are pre-sterilized and placed inside the enclosure 2 before it is sealed. As can be seen in FIG. 2, there is an incision site 7 that is a portion of the enclosure. It can have adhesive along its periphery 7a that allows it to be fixed to the skin of the patient. A cover is placed over this adhesive during manufacture of the enclosure. At the time of the procedure the cover can be removed and the enclosure fixed to the skin of the patient, e.g., about the eye as shown in FIG. 2. Once in place, the surgeon can cut through the enclosure with a scalpel on the instrument tray 5 to expose the tissue of the patient where the ocular surgery is to take place.

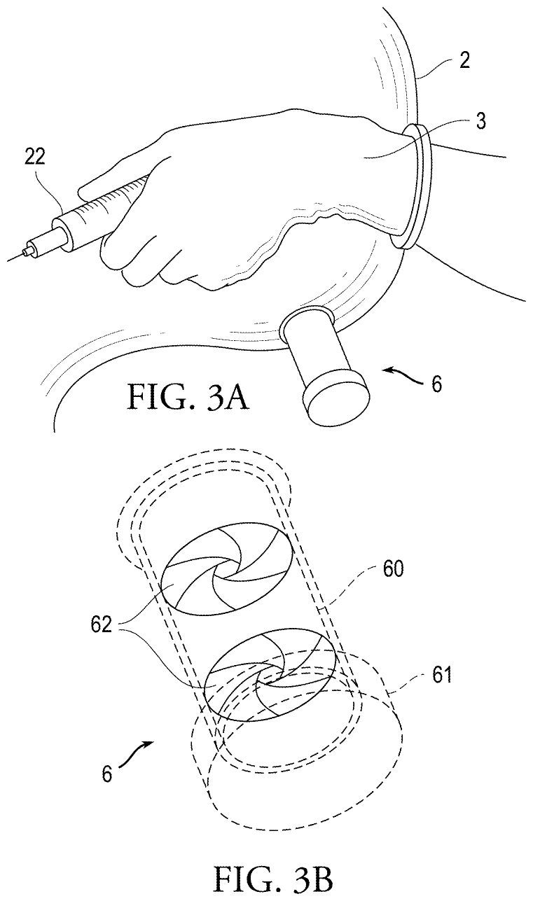

There is a filter 30, such as a HEPA filter, through which air may be drawn into or pushed into the enclosure by a fan or a sterile pump (not shown) to keep the enclosure inflated during the procedure. In one embodiment, the enclosure 2 can be rapidly inflated by using a compressed gas cartridge (not seen) to push air through the filter 30. As an alternative there is an entrance port 6 which is shown enlarged in FIG. 3A that can be used as a way to introduce air into the enclosure from a sterile fan, pump or gas cartridge. FIG. 3B illustrates the details of one embodiment of the entry port 6 formed on the wall of the enclosure 2. The entry port 6 comprises a tubal body 60, a cap 61, and at least two elastic membranes 62. As shown in FIG. 3, the elastic membranes 63 are stacked in the tubal body 60 in the longitudinal direction with a certain interval in which disinfectant solution or gel is filled. After removing the cap 61, the entry port 6 allows for an entry of a sterilized tube or power code into the enclosure 2 while minimizing the risk of contamination.

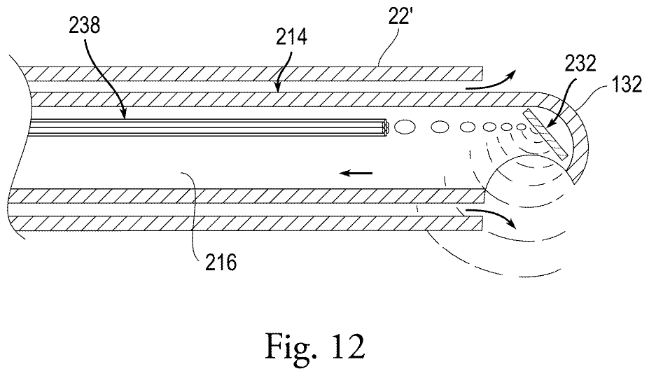

When the enclosure is fully in place and inflated, and the incision site 7 is affixed to the skin of the patient as shown in FIG. 1, the surgeon places his hands in the gloves 3, grapes a scalpel 23 and uses it to remove the portion of the enclosure within the confines of the incision site 7 to expose the underlying tissue of the patient. In a phacoemulsification procedure, an incision is made in the eye by the surgeon to expose the cataract using one of the tools (scalpel) 23 in FIG. 2. Then the surgeon uses the ultrasonic handpiece 22 to breakup the cataract while irrigation fluid is bathing the site from irrigation line 21a and the pieces of cataract and excess fluid are drawn away from the area of the cataract by the aspiration line 21b. If needed or desired, the handpiece can use alternatively use laser energy instead of ultrasonic vibration at the tip of a workpiece 22 to soften, breakup and remove a cataract. For example, the laser light source can be a laser in the handpiece 22′ or a fiber-optic cable 238 extending from a laser outside the enclosure to the handpiece 22′ inside the enclosure as shown in FIG. 12. In this arrangement the fiber-optic cable is located inside a tube 132 and directs laser light pulses to a titanium target 232. The effect of the light pulses hitting the target is to generate shockwaves that emulsify the cataract tissue. A channel 214 is formed between the inner surface of the handpiece 22′ and the outer surface of the tube 132 for carrying irrigation fluid to the operating site. Tissue can be aspirated from the site by a suction force applied to the interior 216 of tube 132.

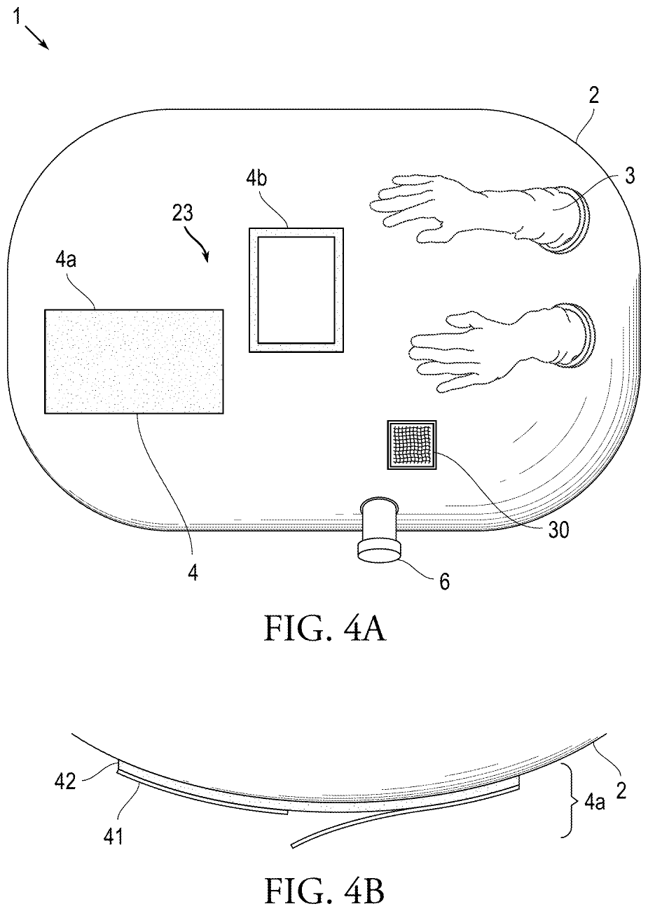

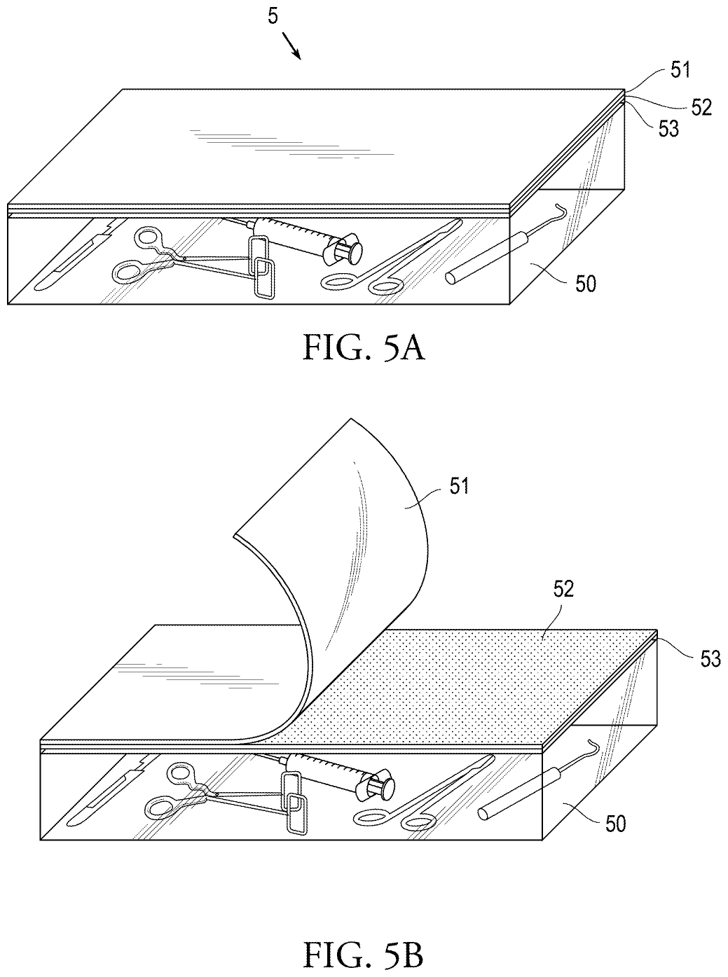

FIG. 4A is a plan view of the surgical sterile field system 1 of the first embodiment. As shown in the drawing, two types of the attachment portions 4 have been formed on the enclosure 2; one 4a is for attaching an instrument tray 5 (FIG. 5A) and another one 4b is for use as an incision site. Although the embodiment shown in FIG. 4A has two attachment portions 4a, 4b, an extra attachment portion 4 may be added if necessary.

As shown in FIG. 4B, the attachment portion 4 has a band-aid type design containing two layers: an adhesive layer 42 disposed on the outer surface of the enclosure 2, and a cover layer 41 disposed on the outer surface of the adhesive layer 42. The cover layer 41 is made of paper or plastic film and can be easily peeled off by hand to expose the adhesive layer 42, to which the instrument tray 5 of FIG. 5A or an incision site on the body of the patient is attached.

As shown in FIGS. 5A & 5B, the instrument tray 5 is composed of a tray box 50 in which sterile medical instruments are store and three layers including a transparent layer 53, an adhesive layer 52, and a cover layer 51 stacked on a top of the instrument tray in this order. Similar to the cover layer 41 of the attachment portion 4 of the enclosure 2, the cover layer 51 of the instrument tray 5 is made of paper or plastic film and can be easily peeled off by hand to expose the adhesive layer 52. The adhesive layer 52 is disposed on the top of the transparent layer 53, which covers the top portion of the tray box 50. In one embodiment, the adhesive layer 52 is also formed transparent.

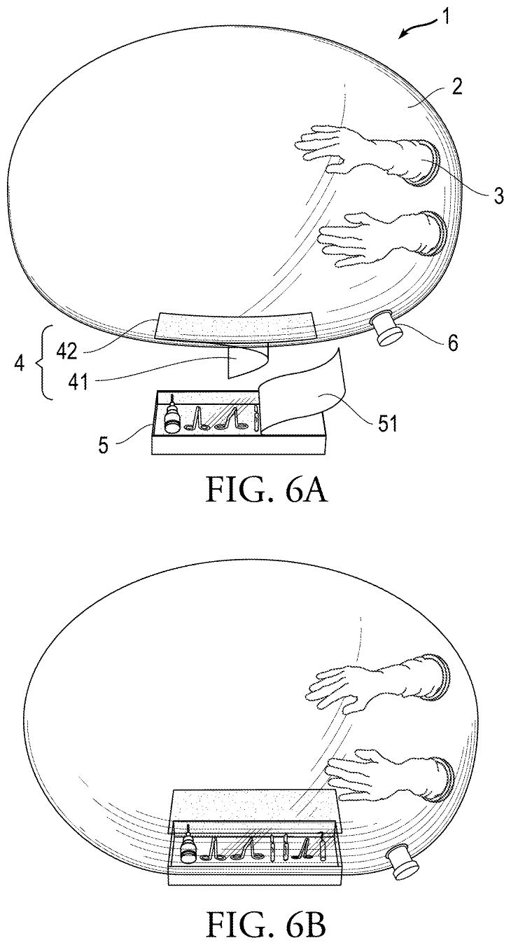

FIGS. 6A-6D illustrate the process of accessing medical instruments in the instrument tray 5 during a surgery. As shown in FIG. 6A, first, the cover layer 41 of the attachment portion 4 of the enclosure 2 and the cover layer 51 of the instrument tray 5 are peeled off. Then, an upper surface of the instrument tray 5 is attached to the adhesive layer 4 of the attachment portion 4 of the enclosure 2. In this embodiment shown in FIG. 6A, the cover layer 41 and the adhesive later 42 of the attachment portion 4 extend along the edges of the attachment portion 4 Likewise, the cover layer 51 and the adhesive layer 52 of the instrument tray 5 at least extends along the edges of the instrument tray 5. Then, the instrument tray 5 is attached to the attachment portion 4 of the enclosure 2 so that the adhesive layer 51 of the instrument tray 5 can be aligned with the adhesive layer 41 of the attachment portion 4.

After attachment, as shown in FIG. 6B, the instrument tray 5 is ready to be accessed by a surgeon. As shown in FIG. 6C, the surgeon inserts his or her hands into the gloves 3 and cuts away the wall of the enclosure 2 together with the adhesive layer 42 of the attachment portion 4 and the adhesive layer 52 and the transparent layer 53 of the instrument tray 5 so that the medical instruments in the instrument tray 5 can be accessed inside the enclosure 2. In some embodiments rather than having the adhesive layer extend along the edges it can cover the entire surface and when access to the tray 5 is needed the surgeon cuts through the layer.

FIG. 6D shows a modified version of the first embodiment where two instrument trays 5 are attached to the enclosure 2. In this manner, the surgeon can access additional sterilized instruments in the tray 5 such as scissors, a syringe, a surgical knife and so on while keeping the surgical field sterile. Thus, the surgery can be efficiently performed while reducing the risk of contamination.

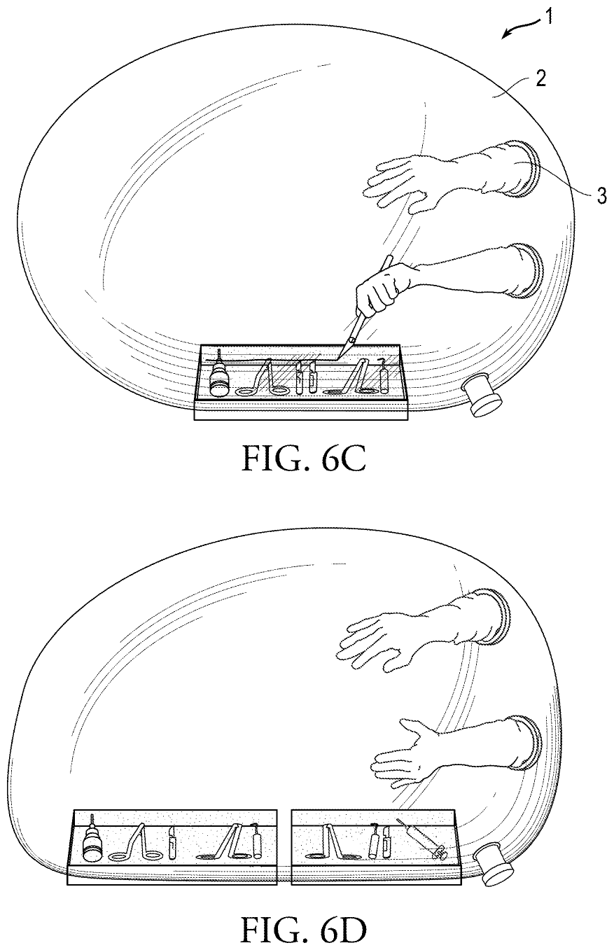

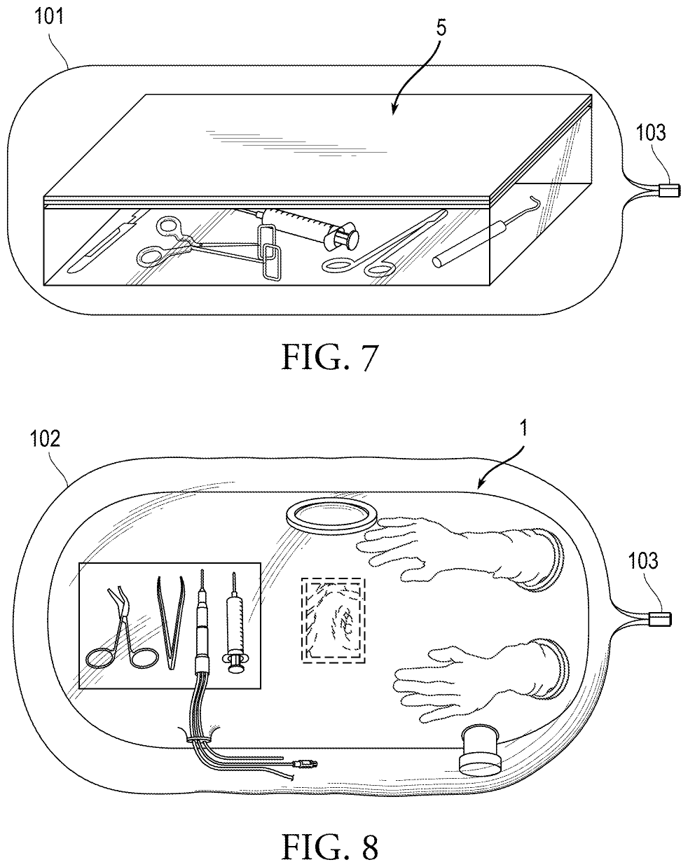

In the present invention, a sterile bag is utilized to store sterilized components. For instance, as shown in FIG. 7, a small sterile bag 101 is used for containing relatively small components such as the instrument tray 5. The sterile bags 101 must be removed before accessing the instrument tray 5. FIG. 8 shows a large sterile bag 102 that is used for containing the entire surgical sterile field system 100. The sterile bag 101, 102 has an opening port 103 that can be pulled apart to open the sterile bag 101, 102.

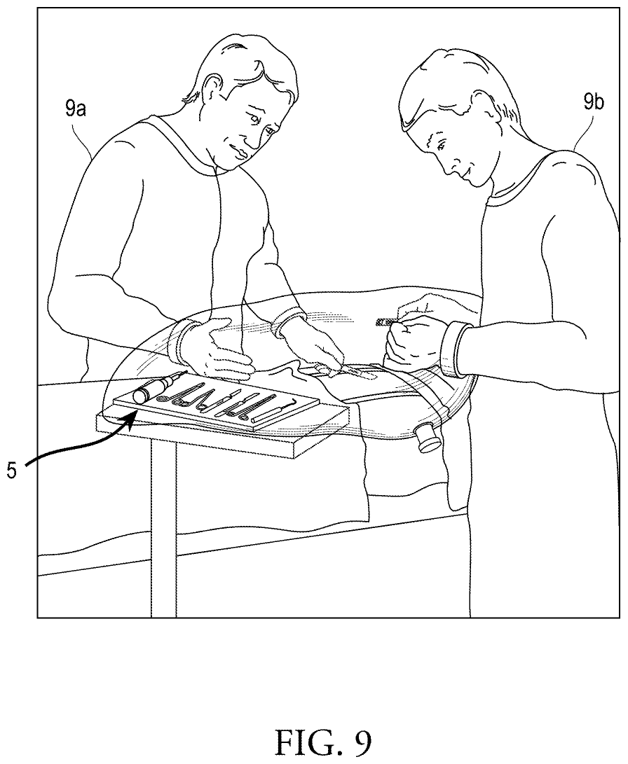

FIG. 9 shows a modified version of the first embodiment where two surgeons 9a, 9b perform general surgery. The surgery may be performed in a hospital or an OBS or a doctor's office using the surgical sterile field system 100 of the present invention. However, the location is not limited to an office, but may include an open field such as a battle field or an accident site. No matter where the operation is performed, thanks to the surgical sterile field system 100, the patient 8 is sufficiently protected from contamination.

As seen in the drawing, two pairs of the integral gloves 3 are provided on opposite side of the wall of the enclosure 2. In addition, the incision site 7 is the chest of the patient. Further, this surgery may be performed without a microscope.

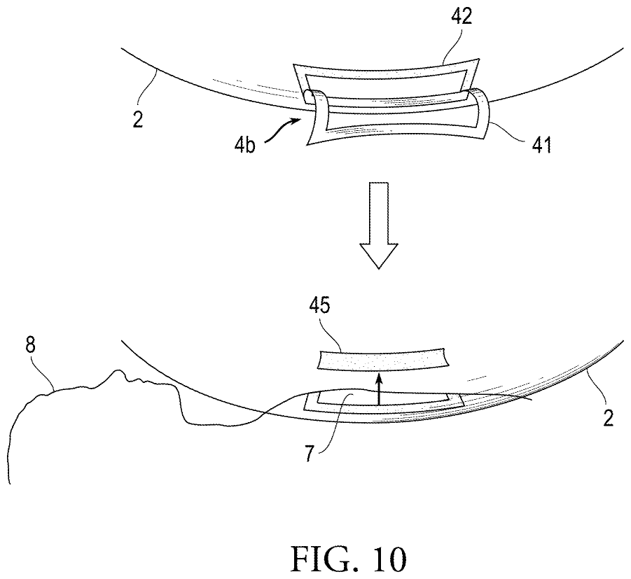

FIG. 10 illustrates the process of attaching the enclosure 2 to the incision site 7. Similar to the process described above, first, the cover layer 41 of the attachment portion of the enclosure 2 is peeled off. Then, the enclosure 2 is lowered so that the adhesive layer of the attachment portion is placed over the incision site 7 of the patient 8. Then, the enclosure 2 is attached directly to the incision site 7. After attachment, the surgeon can simultaneously cut away and remove a portion 45 of the wall of the enclosure 2 and a part of the attachment portion 4 that is surrounded by the adhesive layer 42 so that the incision site 7 is accessible. The removed portion of the enclosure 2 and the attachment portion 4 may be left inside the enclosure 2. In this manner, the surgeon can perform a surgery while minimizing the risk of contamination to the incision site 7. While when performing ocular surgery it is preferable that the adhesive extend about the periphery of the incision site, in general surgery, e.g., chest surgery as shown in FIG. 10, it may be preferable to have the adhesive extend over the entire area and be sealed to the patient's chest. Rather than cut out the portion 45 the surgeon can leave it in place and make an incision directly through the enclosure and adhesive at the incision cite. Once the operation is complete, the wound can even be sutured while the adhesive layer is still adhering to the patient's chest. In such a situation it is beneficial to include an antibiotic or other medicine in the adhesive to guard against infection and to promote healing.

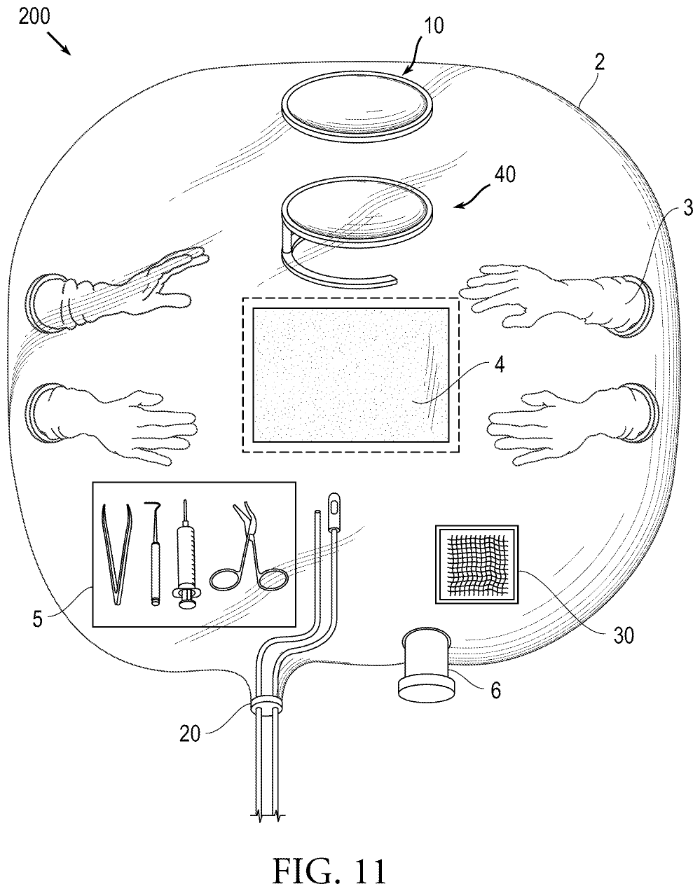

FIG. 11 is the third embodiment of the present invention. The microsurgical sterile field system 200 of the third embodiment differs from that of the second embodiment 100 in the presence of an internal magnifying glass 40 and the number of the integral sterile gloves 3. The magnifying glass 40 is aligned with the incision site 7 and configured to magnify the incision site 7. Further, there can be a modified version of the third embodiment where the exit port is omitted.

According to the surgical sterile field system of the present invention, it is possible to improve the efficiency of surgical procedures in OBS so they can be performed in a doctor's office. Further, it is possible to suppress the cost associated with the procedures since the surgical sterile field system of the present invention does not require a significant amount of sterilization of the operating site. Furthermore, it is possible to reduce the risk of contamination that may occur in regular and OBS procedure rooms. Therefore, the present invention has industrial benefit and applicability.

While preferred embodiments of the invention have been described and illustrated above, it should be understood that these are exemplary of the invention and are not to be considered as limiting. Additions, omissions, substitutions, and other modifications can be made without departing from the spirit or scope of the present invention. Accordingly, the invention is not to be considered as being limited by the foregoing description, and is only limited by the scope of the appended claims.

1. A surgical sterile field system comprising:
an inflatable flexible enclosure that is transparent in at least part of its expanse;
a pair of gloves that are hermetically formed and sealed on a wall of the enclosure to allow access to an inside of the enclosure near a surgical site; and
an incision site of a size corresponding to the required surgical area, said incision site having an adhesive covering at least its exterior periphery, which adhesive is attachable to the skin of the patient adjacent the surgical site, and a peelable cover over the adhesive at the incision site that guards against contact with the adhesive until placed on the patient, wherein upon removal of the cover and application of the incision site of the enclosure to the patient, the enclosure can be cut by a surgical instrument to expose the surgical site while maintaining a sterile environment at the surgical site.

2. The surgical sterile field system according to claim 1, further including
at least two attachment portions formed on an outer surface of the wall of the enclosure, wherein
each of the attachment portions is composed of an adhesive layer and a cover layer stacked on the adhesive layer, and
the cover layer can be manually removed from the adhesive layer.

3. The surgical sterile field system according to claim 1, wherein the enclosure has a filter through which a sterilized air is supplied to inflate the enclosure.

4. The surgical sterile field system according to claim 1,
wherein the enclosure has an entry port formed on the wall thereof, the entry port comprises a tubal body, a cap and at least two elastic membranes; and
wherein items can be entered into the enclosure without causing a lost of sterilization.

5. The surgical sterile field system according to claim 2, further comprising:
an instrument tray that is configured to contain sterilized medical instruments,
wherein one of the at least two attachment portions is configured to attach to the instrument tray, and
another of the at least two attachment portions is configured to directly attach to an incision site of a patient.

6. The surgical sterile field system according to claim 1, wherein the enclosure and the adhesive layer are made of plastic that can be penetrated by a surgical knife.

7. The surgical sterile field system according to claim 5, wherein
the instrument tray comprises a transparent layer, an adhesive layer, and a cover layer stacked on a top of the instrument tray in this order;
the cover layer can be manually removed from the adhesive layer and the adhesive layer of the tray can be mounted on the adhesive layer of the of the one attachment portion, and
a cut can be made through both adhesive layers to allow access to the medical instruments from inside the enclosure.

8. The surgical sterile field system according to claim 7, wherein the transparent layer and the adhesive layer of the instrument tray are made of plastic that can be penetrated by a surgical knife.

9. The surgical sterile field system according to claim 1, further comprising: an additional pair of gloves that are hermetically formed on the wall of the enclosure.

10. The surgical sterile field system according to claim 3, wherein the filter is a HEPA filter.

11. The surgical sterile field system according to claim 1, further comprising:
a microscope connection formed on the wall of the enclosure, and
wherein the microscope connection is configured to attach to a microscope.

12. The surgical sterile field system according to claim 1, further comprising: a magnifying glass located within the enclosure and configured to magnify an incision site of a patient.

13. The surgical sterile field system according to claim 10, wherein
the microscope connection comprises a view window and a rim that surrounds the view window,
the rim is configured to be connected to the microscope and
the view window is configured to be in alignment with an incision site of a patient.

14. The surgical sterile field system according to claim 4, wherein the item is sterile air.

15. The surgical sterile field system according to claim 4, wherein the item is a fluid or power line.

16. The surgical sterile field system according to claim 5, wherein at least one of the medical instruments is an ultrasonic phacoemulsification handpiece.

17. The surgical sterile field system according to claim 5, wherein one of the medical instruments is a laser handpiece that emits laser energy for at least softening a cataract.

18. A method of performing a surgery using the surgical sterile field system according to claim 7, the method comprising the steps of:
inflating the enclosure by supplying sterilized air into the enclosure;
removing the cover layer of the attachment portion to expose the adhesive layer thereof;
removing the cover layer of the instrument tray to expose the adhesive layer thereof;
attaching the instrument tray to the enclosure; and
simultaneously cutting away the wall of the enclosure together with the adhesive layer of the attachment portion and the adhesive layer and the transparent layer of the instrument tray so that the medical instruments in the instrument tray can be exposed inside the enclosure.

19. A method of performing a surgery using the surgical sterile field system according to claim 11, the method comprising the steps of:
inflating the enclosure by supplying sterilized air into the enclosure;
removing the cover layer of the attachment portion to expose the adhesive layer thereof;
removing the cover layer of the instrument tray to expose the adhesive layer thereof;
attaching the instrument tray to the enclosure; and
simultaneously cutting away the wall of the enclosure together with the adhesive layer of the attachment portion and the adhesive layer and the transparent layer of the instrument tray so that the medical instruments in the instrument tray can be exposed inside the enclosure.
